(19) United States
(12) Patent Application Publication
(10) Pub. No.: US 2003/0092656 A1
(43) Pub. Date: May 15, 2003

(54) Therapeutic agents for treatment of solid tumors comprising an expression-inhibiting sustance against Wilms' tumor gene (WT1)

(76) Inventor: Haruo Sugiyama, Mino-shi (JP)

Correspondence Address: FOLEY AND LARDNER, SUITE 500, 3000 K STREET NW, WASHINGTON, DC, 20007, US

(21) Appl. No.: 10/171,639

(22) Filed: Jun. 17, 2002

Related U.S. Application Data
(63) Continuation of application No. 09/462,307, filed on Jan. 6, 2000, now abandoned.
Application No. 09/462,307 is a 371 of international application No. PCT/JP98/03198, filed on Jul. 16, 1998.

(30) Foreign Application Priority Data
Jul. 16, 1997 (JP) 9-191635

(51) Int. Cl. A61K 48/00

(52) U.S. Cl. 514/44

(57) ABSTRACT

The present invention relates to therapeutic agents for treatment of solid tumors comprising an expression-inhibiting substance (an antisense oligonucleotide derivative, a WT1 mutant gene, a WT1 mutant protein, a low molecular weight substance, and the like) against the Wilms' tumor gene (WTI).

### TECHNICAL FIELD

The present invention relates to therapeutic agents for treatment of solid tumors comprising an expression-inhibiting substance against Wilms' tumor gene (WTI).

### BACKGROUND ART

Wilms' tumor is a pediatric kidney tumor resulting from the inactivation of both alleles of the Wilms' tumor gene (WT1) located on chromosome 11p13 (Call K M et al., Cell 60: 509, 1990). The non-coding upstream sequence of WT1 (C. E. Camphell et al., Oncogene 9: 583-595, 1994) and the coding region including introns (D. A. Haber et al., Proc. Natl. Acad. Sci. U.S.A., 88:9618-9622 (1991)) have already been reported, and they are expected to be responsible for the growth and differentiation of tumors and the like (D. A. Haber et al., supra).

Based on the association of WT1 with the growth of leukemia cells, the present inventors have found that an antisense oligonucleotide derivative against WT1 suppresses and/or inhibits the growth of leukemia cells (PCT Patent Publication WO96/38176, and T. Yamagami, et al., Blood, 87(7) 2878-2884 (1996)). It is not known, however, if an expression-inhibiting agent of WT1 suppresses and/or inhibits the growth of solid tumors.

### DISCLOSURE OF THE INVENTION

Thus, the present invention provides a therapeutic agent for treatment of solid tumors comprising an expression-inhibiting substance against Wilms' tumor gene (WTI).

### BRIEF DESCRIPTION OF DRAWINGS

FIG. 1 is a graph showing an inhibitory effect of an oligonucleotide at 100 μg/ml on the cellular growth of the gastric cancer AZ521 cell line.

FIG. 2 is a graph showing an inhibitory effect of an oligonucleotide at 200 μg/ml on the cellular growth of the gastric cancer AZ521 cell line.

FIG. 3 is a graph showing an inhibitory effect of an oligonucleotide at 400 μg/ml on the cellular growth of the gastric cancer AZ521 cell line.

FIG. 4 is a graph showing an inhibitory effect of an oligonucleotide at 200 μg/ml on the cellular growth of the lung cancer OS3 cell line.

FIG. 5 is a graph showing an inhibitory effect of an oligonucleotide at 400 μg/ml on the cellular growth of the lung cancer OS3 cell line.

FIG. 6 is a graph showing an inhibitory effect of an oligonucleotide at 400 μg/ml on the cellular growth of the ovary cancer TYKnu cell line.

FIG. 7 is a graph showing an inhibitory effect of an oligonucleotide at 400 μg/ml on the cellular growth of WTAS PC14, a WT1-expression-negative lung adenocarcinoma cell line.

### BEST MODE FOR CARRYING OUT THE INVENTION

The present invention provides a therapeutic agent, for treatment of solid tumors, comprising an expression-inhibiting substance against WTI. Solid tumors, as used herein, mean, for example, gastric cancer, colon cancer, lung cancer, breast cancer, embryonic cell cancer, hepatic cancer, skin cancer, cystic cancer, prostate cancer, uterine cancer, cervical cancer, ovary cancer, and the like. The expression-inhibiting substance for use in the present invention may be any substance that inhibits the expression of WT1 and includes, for example, an antisense oligonucleotide derivative against WT1, a low molecular weight inhibiting substance such as a WT1 mutant gene, a mutant protein and decoy DNA etc. that act on WT1 in a dominant negative manner, or a low molecular weight inhibiting substance such as a peptide that inhibits transcription activity by specifically binding to WT1, and the like. The antisense oligonucleotide derivative for use in the present invention may be an antisense oligonucleotide derivative against WT1 including, for example, one against the transcription capping site of WT1, one against the translation initiation region, an exon or an intron, and the like.

For example, a nucleotide sequence of a sense DNA strand in a region containing the transcription capping site of WT1 is represented by SEQ ID NO: 9 and nucleotide sequences of the sense DNA strands of exon 1 to 10 in the coding region of WT1 are represented by SEQ ID NO: 10 to 19, respectively. The present invention employs antisense oligonucleotide derivatives against such nucleotide sequences of the sense DNA strands of WT1. The antisense oligonucleotide derivative is an antisense oligonucleotide derivative comprising 5 to 50, preferably 9 to 30 contiguous nucleotides of an antisense DNA strand or an RNA strand of WT1, or 5 to 70, preferably 9 to 50 intermittently or partially complementary nucleotides, provided that it can bind to the DNA strand or the RNA strand of WT1.

As the one against a transcription capping site, there may be mentioned the following nucleotide sequences: 5′-AGGGTCGAATGCGGTGGG-3′ (SEQ ID NO: 2) and 5′-TCAAATAAGAGGGGCCGG-3′ (SEQ ID NO: 4). Furthermore, as the one against a translation initiation region, there may be mentioned antisense oligonucleotide derivatives against the translation initiation codon ATG and a region containing the upstream and/or the downstream thereof including, for example, the following nucleotide sequence: 5′-GTCGGAGCCCATTTGCTG-3′ (SEQ ID NO: 6).

The coding region of WT1 contains 10 exons. The antisense oligonucleotide derivative of the present invention is directed against a sequence contained in any of these exons, a sequence covering any of two contiguous exons after splicing, a sequence covering a contiguous intron and exon, and a sequence of any intron and 3′ or 5′-end non-coding region. One example is against the sixth exon that has the following sequence: 5′-CGTTGTGTGGTTATCGCT-3′ (SEQ ID NO: 8).

Furthermore, the region corresponding to the antisense oligonucleotide derivative of the present invention having a nucleotide sequence that is intermittently or partially complementary to the DNA strand or the RNA strand of WT1 includes, but not limited to, a ribozyme having a function of cleaving any region of a DNA strand or of an RNA strand of WT1.

The structure of the antisense oligonucleotide derivative for use in the present invention is as shown in Chemical formula (1) wherein X may be independently any of oxygen (O), sulfur (S), a lower alkyl group and a primary amine or a secondary amine; Y may be independently any of oxygen (O) and sulfur (S); Z is hydrogen or a hydroxyl group; when Z is hydrogen B is selected from the group consisting of adenine, guanine, thymine and cytosine, and when Z is a hydroxyl group B is selected from the group consisting of adenine, guanine, uracil and cytosine, and B is primarily a complementary oligonucleotide to the DNA or the mRNA encoding WT1; R is independently hydrogen or a dimethoxytrityl group or a lower alkyl group; and n is 7 to 28.

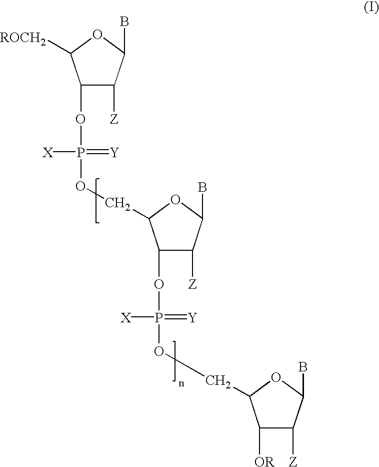

Preferred antisense oligonucleotide derivatives include not only non-modified antisense oligonucleotides but also modified antisense oligonucleotides. Examples of such modifications include, for example, lower alkyl phosphonate-modifications such as the above-mentioned methylphosphonate type or the ethylphosphonate type, and the phosphorothioate modifications or the phosphoroamidate modifications (see Chemical formula (2)).

Examples of

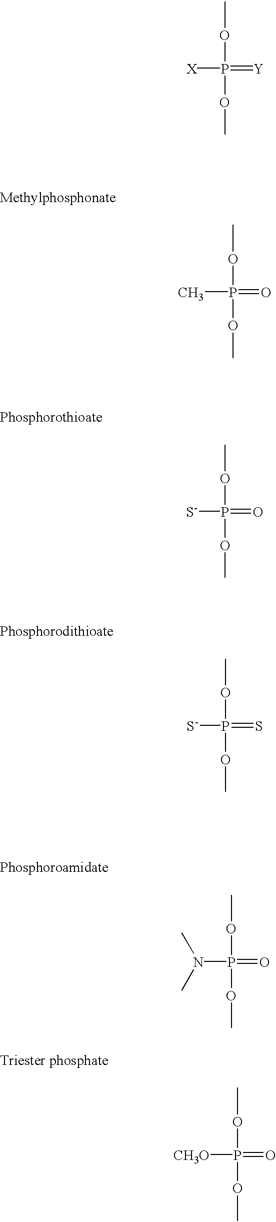

These antisense oligonucleotide derivatives can be obtained by a conventional method as shown below.

An antisense oligonucleotide of Formula (1) in which X and Y are O and Z is hydrogen or a hydroxyl group may be readily synthesized using a commercially available DNA synthesizer (for example the one manufactured by Applied Biosystems).

Synthesis of an antisense oligodeoxy ribonucleotide in which Z is hydrogen can be effected by the solid phase synthesis using phosphoroamidite, the solid phase synthesis using hydrogen phosphonate, or the like.

See, for example, T. Atkinson, M. Smith, in Oligonucleotide Synthesis: A Practical Approach, ed. M. J. Gait, IRL Press, 35-81 (1984); M. H. Caruthers, Science, 230, 181 (1985); A. Kume, M. Fujii, M. Sekine, M. Hata, J. Org. Chem., 49, 2139 (1984); B. C. Froehler, M. Matteucci, Tetrahedron Lett., 27, 469 (1986); P. J. Garegg, I. Lindh, T. Regberg, J. Stawinski, R. Stromberg, C. Henrichson, ibid, 27, 4051 (1986); B. S. Sproat, M. J. Gait, in Oligonucleotide Synthesis: A Practical Approach, ed. M. J. Gait, IRL Press, 83-115 (1984); S. L. Beaucage and M. H. Caruthers, Tetrahedron Lett., 22, 1859-1862 (1981); M. D. Matteucci and M. H. Caruthers, Tetrahedron Lett., 21, 719-722 (1980); M. D. Matteucci and M. H. Caruthers, J. Am. Chem. Soc., 103, 3185-3191 (1981)

A triester phosphate modification derivative in which X is a lower alkoxy group can be obtained by, for example, a conventional method in which an oligonucleotide that was obtained by chemical synthesis is treated with a solution of tosyl chloride in DMF/methanol/2,6-lutidiene (Moody H. M. et al., Nucleic Acids Res., 17, 4769-4782 (1989)).

An alkyl phosphonate modification derivative in which X is an alkyl group can be obtained by, for example, using phosphoamidite (M. A. Dorman, et al., Tetrahedron, 40, 95-102 (1984); K. L. Agarwal and F. Riftina, Nucleic Acids Res., 6, 3009-3024 (1979)).

A triester phosphorothioate modification derivative in which X is S can be obtained by a solid phase synthesis using sulfur (C. A. Stein, et al., Nucleic Acids Res., 16, 3209-3221 (1988)), or a solid phase synthesis using tetraethyltiraum disulfide (H. Vu and B. L. Hirschbein, Tetrahedron Letters, 32, 3005-3008 (1991).

A phosphorodithioate modification derivative in which both X and Y are S can be obtained by, for example, a solid phase synthesis in which a bisamidite is converted to a thioamidite, to which is added sulfur to yield said modification (W. K. -D. Brill, et al., J. Am Chem. Soc., 111, 2321-2322 (1989)).

A phosphoroamidate modification derivative in which X is a primary amine or a secondary amine can be obtained by, for example, a solid phase synthesis in which hydrogen phosphonate is treated with a primary or secondary amine (B. Froehler, et al., Nucleic Acids Res., 16, 4831-4839 (1988)). Alternatively the amidite may be oxidized with tert-butyl hydroperoxide to yield said modification (H. Ozaki, et al., Tetrahedron Lett., 30, 5899-5902 (1989)).

Although the synthetic method of an antisense oligoribonucleotide in which Z is a hydroxyl group is very complicated as compared to that of an antisense oligodeoxyribonucleotide in that a 2′-hydroxyl group present in the ribose (sugar) must be protected in the former method, it can be synthesized by selecting, as appropriate, the protecting group and the method of phosphorylation (see Biseibutugaku Kiso Koza (Basic Course in Microbiology), Vol. 8, Eiko Ohtsuka and Kazunobu Miura, Idenshi Kogaku (Genetic Engineering), Tadahiko Ando and Kenji Sakaguchi ed., Oct. 10, 1987, Kyoritsu Shuppan Publishing Company).

Purification and the confirmation of purity can be carried out by high performance liquid chromatography and polyacrylamide gel electrophoresis. The confirmation of molecular weight can be carried out by Electrospray Ionization Mass Spectrometry or Fast Atom Bombardment-Mass Spectrometry.

The expression-inhibiting substance against WT1 of the present invention is believed to inhibit the growth of solid tumor cells by acting in any stage from genomic DNA to mature mRNA and by inhibiting the expression thereof. Thus, the expression-inhibiting substance of the present invention is expected to be useful for the treatment of solid tumors.

The expression-inhibiting substance of the present invention can be mixed with an appropriate carrier material to formulate an external preparation such as a liniment, a cataplasm and the like.

It can also be mixed, as desired, with an excipient, an isotonic agent, a solubilizer, a stabilizer, an antiseptic, a soothing agent or the like to formulate a tablet, powder, granules, a capsule, a liposome capsule, an injection, a solution, a nasal drop, and the like as well as a lyophilized preparation. They can be prepared according to a conventional method.

The expression-inhibiting substance of the present invention may be applied to the patient by either directly administering it to the affected area of the patient or administering it into a blood vessel thereby allowing the substance to be delivered to the affected area. Furthermore, an encapsulating agent that enhances prolonged action and membrane permeability may be used. There may be mentioned, for example, liposome, poly-L-lysine, lipid, cholesterol, lipofectin or derivatives thereof.

Preferably the dosage of the expression-inhibiting substance of the present invention can be adjusted as appropriate depending on the condition, age, sex, weight, and the like of the patient to employ a preferred amount. The method of administration may be selected, as appropriate, from oral, intramuscular, intraperitoneal, intrathoracic, intraspinal, intratumoral, intradermal, subcutaneous, intravenous, intraarterial, rectal administration and the like to employ a preferred method.

The present invention is now explained in more detail with reference to the following examples.

### EXAMPLES

### Synthetic Example 1

The oligodeoxyribonucleotides (SEQ ID NO: 1 to 8) and the random sequence (Rand) used below were synthesized using an automatic synthetic instrument (Applied Biosystems), which were then purified by high performance liquid chromatography, were subjected to ethanol precipitation for three times, and then were suspended in a phosphate buffered saline.

The oligonucleotides that were synthesized are shown below. The random sequence (Rand) is a sequence comprising 18 nucleotides and thereby is theoretically a mixture of sequences of 4 to the 18th power kinds.

SEQ ID NO: 1: A sense sequence of the transcription capping site (SE1);

SEQ ID NO: 2: An antisense sequence of the transcription capping site (AS1);

SEQ ID NO: 3: A sense sequence of the transcription capping site;

SEQ ID NO: 4: An antisense sequence of the transcription capping site;

SEQ ID NO: 5: A sense sequence of the translation initiation region (SE2);

SEQ ID NO: 6: A antisense sequence of the translation initiation region (AS2);

SEQ ID NO: 7: A sense sequence of exon 6;

SEQ ID NO: 8: An antisense sequence of exon 6;

### Example 1

Cells of the WT1 expression-positive gastric cancer AZ521 cell line at 5×10⁴ cells/ml were inoculated at an amount of 100 μl/well into the RPMI1640 medium containing no fetal calf serum (FCS) in a flat-bottomed 96-well plate. The oligonucleotide AS1 or the control SE1 or rand was added to triple wells to a final concentration of 100 μg/ml. After incubation for 2 hours, FCS was added to each well to a final concentration of 10%. The oligonucleotide of half the above amount was added to the culture every 24 hours.

After incubation for 96 hours, the surviving cells were counted by the dye exclusion method. As a control culture, PBS having the same volume containing no nucleotides was added and the cell count in this culture was set as 100%.

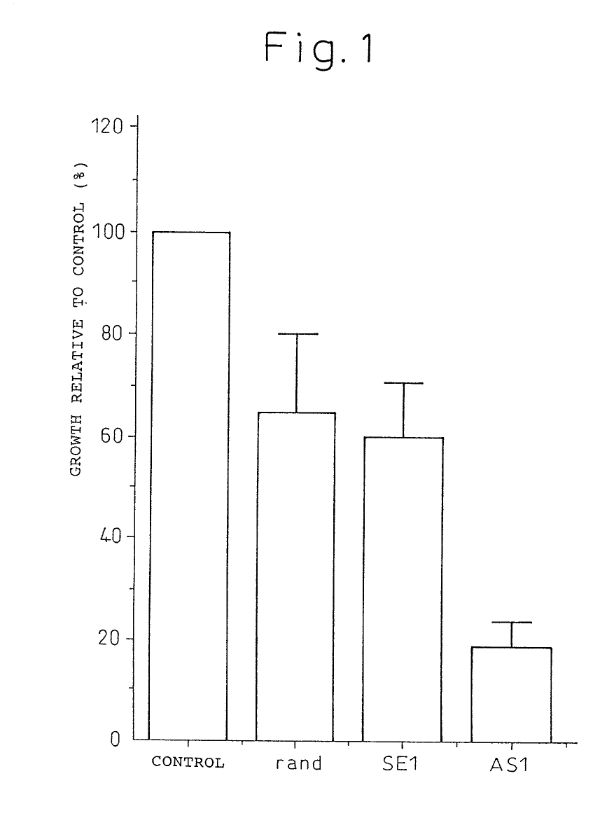

The result is shown in FIG. 1. As can be seen from this figure, the antisense oligonucleotide AS1 of the present invention strongly inhibited the growth of cells as compared to the corresponding sense oligonucleotide SE1.

### Example 2

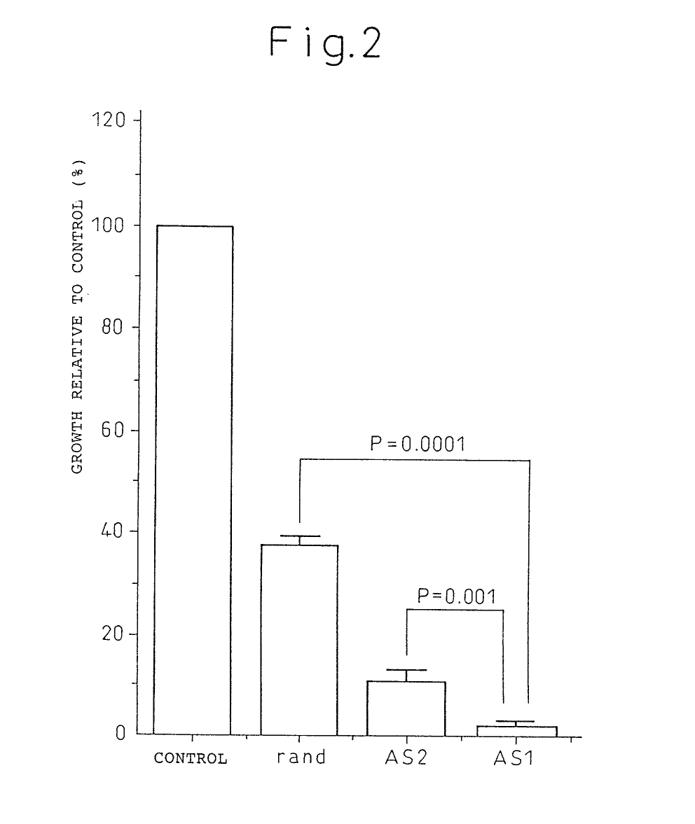

A similar experiment to the one described in Example 1 was carried out, except that the oligonucleotide AS1 or AS2, or rand was added at 200 μg/ml. As can be seen from FIG. 2, the antisense oligonucleotides AS1 and AS2 of the present invention significantly inhibited the growth of gastric cancer cells as compared to the random sequence (rand).

### Example 3

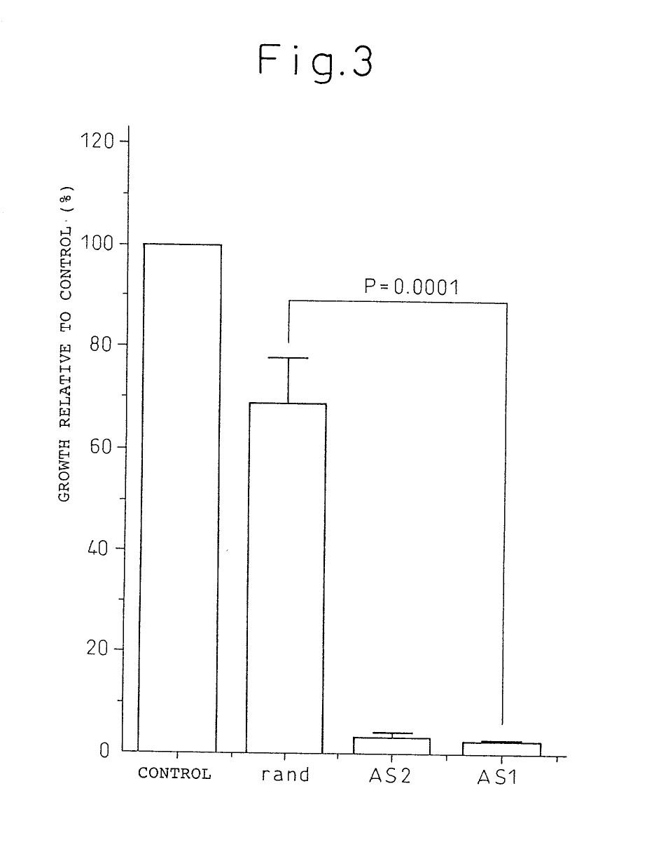

A similar experiment to the one described in Example 1 was carried out, except that the oligonucleotide AS1 or AS2, or rand was added at 400 μg/ml. As can be seen from FIG. 3, the antisense oligonucleotides AS1 and AS2 of the present invention significantly inhibited the growth of gastric cancer cells as compared to the random sequence (rand).

As is clear from the results in Examples 1 to 3, the inhibitory effect of the antisense oligonucleotide of the present invention on the growth of the gastric cancer cells was concentration-dependent.

### Example 4

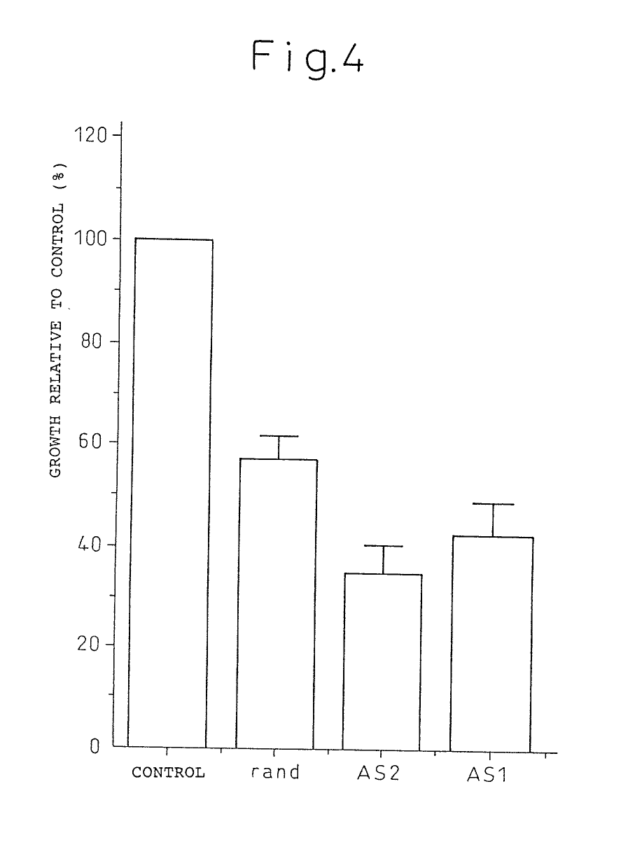

A similar experiment to the one described in Example 1 was carried out, except that cells of the lung cancer OS3 cell line were used as solid tumor cells, and the antisense oligonucleotide AS1 or AS2 or the random sequence (rand) was used at 200 μg/ml. As can be seen from FIG. 4, the antisense oligonucleotides AS1 and AS2 of the present invention significantly inhibited the growth of the lung cancer cells as compared to the random sequence (rand).

### Example 5

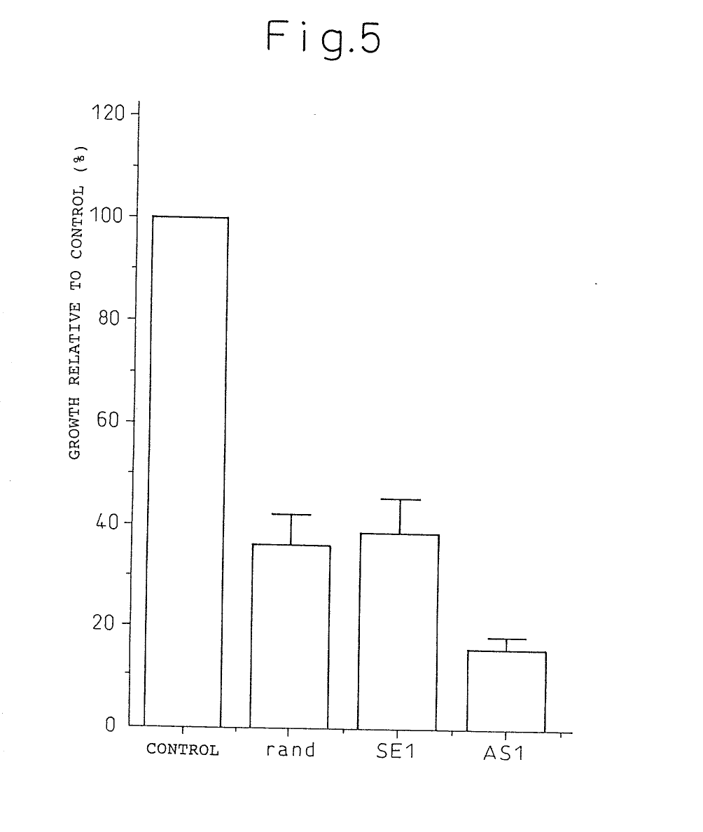

A similar experiment to the one described in Example 1 was carried out, except that the antisense oligonucleotide AS1 at 400 μg/ml or SE1 or rand at 400 μg/ml as a control were used. AS can be seen from FIG. 5, the antisense oligonucleotide AS1 of the present invention significantly inhibited the growth of lung cancer cells as compared to the other control oligonucleotides.

As is clear from the comparisons in Examples 4 and 5, the inhibitory effect of the antisense oligonucleotide of the present invention on the growth of the lung cancer cells was concentration-dependent.

### Example 6

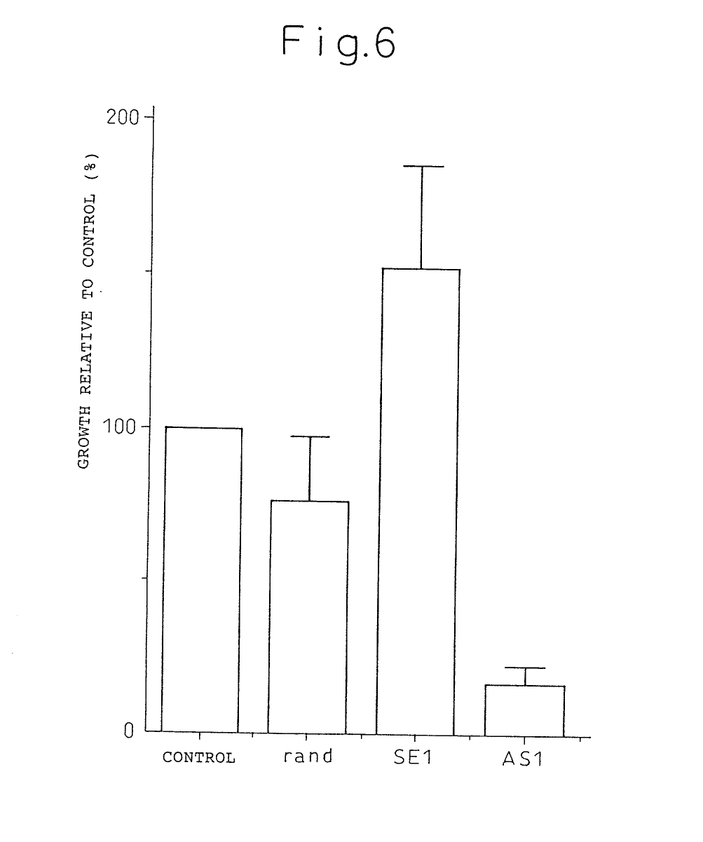

A similar experiment to the one described in Example 1 was carried out, except that the cells of the ovary cancer TYKnu cell line were used, the antisense oligonucleotide AS1 at 400 μg/ml or SE1 or the control oligonucleotide SE1 or rand at 400 μg/ml were used. As can be seen from FIG. 6, the antisense oligonucleotide AS1 of the present invention has shown a marked inhibitory effect on ovary cancer cells as compared to the control oligonucleotide.

### Reference Example 1

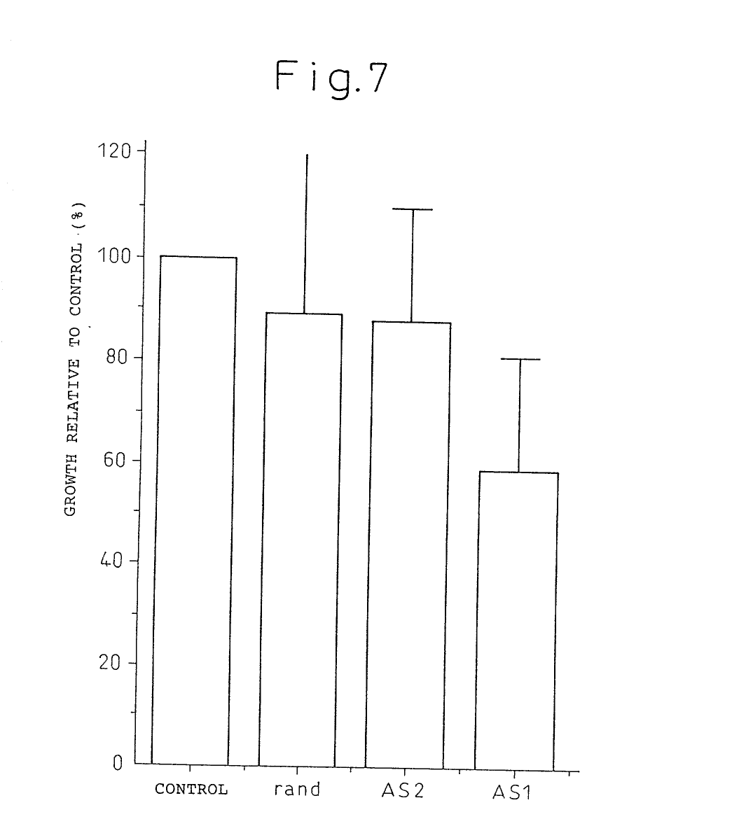

A similar experiment to those described in Examples was carried out, except that the cells of the WT1 expression-negative lung adenocarcinoma cell line WTAS PC14 were used as the test cells, the antisense oligonucleotide AS1 or AS2 at 400 μg/ml or the control oligonucleotide rand at 400 μg/ml were used. As can be seen from FIG. 7, the antisense oligonucleotide of the present invention did not exhibit a marked inhibitory effect on growth as compared to the WT1 expression-positive cells.

### Example 7

RNA was extracted from each cancer cell line shown in Table 2 and the amount expressed of WT1 mRNA was determined using the RT-PCR method described below. The amount expressed of WT1 in the leukemia cell line K562 was set as 1.0 and the amount expressed of WT1 in each cancer cell line was shown at a relative amount in Table 2.

Total RNA from each cell line was extracted according to the conventional method [for example, the acid-guanidine-phenol-chloroform method: Anal. Biochem., 162, 156 (1987)]. The total RNA was dissolved in a diethyl pyrocarbonate-treated water, and then the absorbance at 260 nm was spectrophotometrically determined.

15.5 μl of the diethyl pyrocarbonate-treated water containing 1 μg of the total RNA was heated at 65° C. for 5 minutes, and was mixed with 14.5 μl of the RT buffer (50 mmol/l Tris HCl, pH 8.3; 70 mmol/l KCl; 3 mmol/l MgCl₂; 10 mmol/l dithiothreitol) containing 600 U of a reverse transcriptase (Moloney murine leukemia virus reverse transcriptase: GIBCO-BRL), 500 mmol/l of each deoxynucleotide triphosphate (dNTP: Pharmacia) and 750 ng of an olido dT primer and 40 U of an RNase inhibitor (Boehringer Mannheim).

The mixture was incubated at 37° C. for 90 minutes and heated at 70° C. for 20 minutes, and then was stored at −20° C. until use.

PCR was conducted using a DNA thermal cycler (Perkin Elmer-Cetus) at repeated cycles of denaturation at 94° C. for 1 minute, primer annealing at 64° C. for 1 minute (β actin: 60° C., 1 minute), and chain elongation at 72° C. for 2 minutes to obtain a PCR product (the first round PCR).

When the densitomer unit (described below) of said PCR product is less than 500, the second round PCR was carried out using nested inward primers in a reaction mixture comprising 2.5 μl of the first round PCR product.

The PCR product thus obtained was determined according to the method described in an article [J. Immunol., 147, 4307 (1991)] as described below:

Thus, the PCR product from 20 ng of total RNA was resolved on a 1.3% agarose gel containing 0.05 μg/ml ethidium bromide, and photographed with a Polaroid film (Polaroid 665 film, Polaroid Corp.).

The negative film was developed at 25° C. for 5 minutes and was assayed with a densitometer (CS-9000: Shimadzu) to obtain “densitometer units”.

Furthermore, the result obtained from the above experiment using the PCR product in the absence of RNA was set as the negative control.

The primers used in the above experiment are as shown in Table 1.

TABLE 1

Nucleotide sequence

| | |
|---|---|
| First round PCR | |
| primer | |
| Outward sense primer | 5′-GGCATCTGAGACCAGTGAGAA-3′ |
| | (SEQ ID NO:20) |
| Outward antisense | 5′-GAGAGTCAGACTTGAAAGCAGT-3′ |
| primer | (SEQ ID NO:21) |
| Second round PCR | |
| primer | |
| Inward sense primer | 5′-GCTGTCCCACTTACAGATGCA-3′ |
| | (SEQ ID NO:22) |
| Inward antisense | 5′-TCAAAGCGCCAGCTGGAGTTT-3′ |
| primer | (SEQ ID NO:23) |

As primers for the β actin that was used as an internal control, those described in an article [Proc. Natl. Acad. Sci. U.S.A. 82, 6133 (1985)] were used. Each of these primers was chemically synthesized according to a conventional method.

In order to standardize the differences in the amount used of RNA in RT-PCR and RNA degradation in each sample, the result of the WT1 gene (densitometer units) was divided by that of β actin, which was set as the level of the WT1 gene expression.

The result is shown in Table 2.

TABLE 2

| Origin | Cell line | Amount expressed of WT |
|---|---|---|
| Gastric cancer | AZ 521 | 1.2 × 10⁰ |
| Colon cancer | LOVO | 1.1 × 10⁻³ |
| | SW 480 | 2.3 × 10⁻¹ |
| | SW 620 | 1.0 × 10⁻¹ |
| | COLO 320 DM | 7.3 × 10⁻³ |
| Lung cancer | OS 1 | 1.6 × 10⁻² |
| | OS 2R | 8.3 × 10⁻³ |
| | OS 3 | 3.1 × 10⁻² |
| | LU 99B | 2.9 × 10⁻² |
| | LU 99C | 3.4 × 10⁻² |
| | VMRC-LCP | 4.9 × 10⁻¹ |
| Breast cancer | MDA MB 231 | 3.3 × 10⁻² |
| | YMB 1 | 5.2 × 10⁻² |
| Embryonic cell | NEC 8 | 5.8 × 10⁻³ |
| cancer | | |
| Ovary cancer | TYK NU | 4.5 × 10⁻¹ |
| | TYK nu. CP-r | 2.5 × 10⁻¹ |
| Leukemia | K 562 | 1.0 × 10⁻⁰ |
| (control) | | |

The above result confirmed that the WT1 gene is expressed in the cultured cell lines derived from various solid tumors.

As hereinabove stated, the antisense oligonucleotides of the present invention are useful for inhibiting the growth of solid tumor cells and thereby are expected to be novel therapeutic agents for treatment of solid tumors.

1. A therapeutic agent for treatment of solid tumors comprising an expression-inhibiting substance against Wilms' tumor gene (WTI).

2. The therapeutic agent according to claim 1 for treatment of solid tumors, wherein said expression-inhibiting substance is an antisense oligonucleotide derivative.

3. The therapeutic agent according to claim 1 for treatment of solid tumors, wherein said expression-inhibiting substance is the WT1 mutant gene.

4. The therapeutic agent according to claim 1 for treatment of solid tumors, wherein said expression-inhibiting substance is a WT1 mutant protein.

5. The therapeutic agent according to claim 1 for treatment of solid tumors, wherein said expression-inhibiting substance is a low molecular weight substance.

6. The therapeutic agent according to claim 2 for treatment of solid tumors, wherein said antisense oligonucleotide derivative is an antisense oligonucleotide against at least 9 contiguous nucleotides of a transcription capping site of the Wilms' tumor gene.

7. The therapeutic agent according to claim 6 for treatment of solid tumors, wherein said antisense oligonucleotide derivative has the nucleotide sequence:
5′-AGGGTCGAATGCGGTGGG-3′ (SEQ ID NO: 2) or
5′-tcaaataagaggggccgg-3′ (SEQ ID NO: 4).

8. The therapeutic agent according to claim 2 for treatment of solid tumors, wherein said antisense oligonucleotide derivative is an antisense oligonucleotide against at least 9 contiguous nucleotides of the translation initiation region of the Wilms' tumor gene.

9. The therapeutic agent according to claim 8 for treatment of solid tumors, wherein said antisense oligonucleotide has the nucleotide sequence:
5′-GTCGGAGCCCATTTGCTG-3′ (SEQ ID NO: 6).

10. The therapeutic agent according to claim 2 for treatment of solid tumors, wherein said antisense oligonucleotide derivative is an antisense oligonucleotide against at least 9 contiguous nucleotides of an exon of the Wilms' tumor gene.

11. The therapeutic agent according to claim 10 for treatment of solid tumors, wherein said exon is exon 6.

12. The therapeutic agent according to claim 11 for treatment of solid tumors, wherein said antisense oligonucleotide derivative has the nucleotide sequence:
5′-CGTTGTGTGGTTATCGCT-3′ (SEQ ID NO: 8).

13. The therapeutic agent according to any of claims 1 to 12 for treatment of solid tumors, wherein said solid tumor is gastric cancer, colon cancer, lung cancer, breast cancer, embryonic cell cancer, hepatic cancer, skin cancer, cystic cancer, prostate cancer, uterine cancer, cervical cancer, or ovary cancer.
